(19) United States
(12) Patent Application Publication
(10) Pub. No.: US 2016/0151298 A1
(43) Pub. Date: Jun. 2, 2016

(54) DOCETAXEL POLYMERIC NANOPARTICLES AND METHODS OF TREATING CANCERS USING SAME

(71) Applicant: BIND THERAPEUTICS, INC., Cambridge, MA (US)

(72) Inventor: James Wright, Lexington, MA (US)

(21) Appl. No.: 14/900,925

(22) PCT Filed: Jun. 27, 2014
(86) PCT No.: PCT/US2014/044617

§ 371 Date: Dec. 22, 2015

Related U.S. Application Data
(60) Provisional application No. 61/871,453, filed on Aug. 29, 2013.
(60) Provisional application No. 61/840,950, filed on Jun. 28, 2013.

(51) Int. Cl. A61K 9/51; A61K 47/26; A61K 9/10; A61K 31/337; A61K 9/00

(52) U.S. Cl. CPC A61K 9/5153; A61K 31/337; A61K 9/0019; A61K 9/10; A61K 47/26

(57) ABSTRACT

The present disclosure generally relates to suspensions and compositions of polymeric nanoparticles that include docetaxel, as well as methods of treating various cancers, including refractory or drug resistant cancers in patients in need thereof using disclosed compositions.

### CROSS REFERENCE TO RELATED APPLICATIONS

This application claims the benefit of and priority to U.S. Provisional Patent Application No. 61/840,950, filed Jun. 28, 2013, and U.S. Provisional Patent Application No. 61/871,453, filed Aug. 29, 2013, each of which is hereby incorporated by reference in its entirety.

### BACKGROUND

A variety of cancers are described in detail in the medical literature. Examples include bladder cancer, brain cancer, breast cancer, cervical cancer, colon cancer (including colorectal cancer), esophageal cancer, head and neck cancer, prostate cancer, liver cancer, lung cancer (both small cell and non-small cell), melanoma, myeloma, neuroblastoma, ovarian cancer, pancreatic cancer, prostate cancer, renal cancer, sarcoma (including osteosarcoma), skin cancer (including squamous cell carcinoma), stomach cancer, testicular cancer, thyroid cancer, uterine cancer, and hematologic cancers. The incidence of cancer continues to climb as the general population ages, and as new cancers develop. However, options for the treatment of cancer are limited. For example, may cancers have few treatment options available, especially when one or multiple courses of conventional chemotherapy fail.

Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols.

Therapeutics that include an active drug and that are e.g., targeted to a particular tissue or cell type or targeted to a specific diseased tissue but not to normal tissue, may reduce the amount of the drug in tissues of the body that are not targeted and may be more effective and less toxic. This is particularly important when treating a condition such as cancer where it is desirable that a cytotoxic dose of the drug is delivered to cancer cells without killing the surrounding non-cancerous tissue. Effective drug targeting may reduce the undesirable and sometimes life threatening side effects common in anticancer therapy. In addition, such therapeutics may allow drugs to reach certain tissues they would otherwise be unable to reach.

Therapeutics that offer controlled release and/or targeted therapy also must be able to deliver an effective amount of drug, which is a known limitation in other nanoparticle delivery systems. For example, it can be a challenge to prepare nanoparticle systems that have an appropriate amount of drug associated each nanoparticle, while keeping the size of the nanoparticles small enough to have advantageous delivery properties.

Accordingly, a need exists for nanoparticle therapeutics and methods of making such nanoparticles, that are capable of delivering therapeutic levels of drug, for example, higher levels of drug, to treat diseases such as cancer, while also reducing patient side effects especially at when higher levels necessary for effective treatment are administered. There is a significant need for safe and effective methods of treating, preventing and managing cancer and other diseases and conditions, particularly for diseases that are refractory to standard treatments, such as surgery, radiation therapy, chemotherapy and hormonal therapy, while reducing or avoiding the toxicities and/or side effects associated with the conventional therapies.

### SUMMARY

The present disclosure generally relates to suspensions and compositions of polymeric nanoparticles that include docetaxel, as well as methods of treating various cancers, including refractory or drug resistant cancers in patients in need thereof using disclosed compositions.

In one aspect, a method of treating cancer, or a refractory cancer in patient in need thereof is provided. The method comprises intravenously administering to the patient an effective amount of a therapeutic nanoparticle suspension, comprising:

a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa with a pentylene end group, wherein the pentylene end group is conjugated through an amide linkage to the moiety S,S-2-{3-[1-carboxy-5-amino-pentyl]-ureido}-pentanedioic acid; and
a surfactant; and

an aqueous suspending medium, wherein the suspension is administered once every week, every two weeks, every three weeks, or every four weeks.

Contemplated methods, in some embodiments, include administering to the patient a disclosed suspension once every week, for example, in a dose (e.g. a weekly dose) of about 15 mg/m² to 50 mg/m² or more, or about 30 mg/m² to about 50 mg/m² or more of docetaxel.

In a particular embodiment, a cumulative maximum tolerated dose of docetaxel is greater when disclosed suspensions are administered weekly as compared to administering the suspension every three weeks. For example, in certain embodiments, the cumulative maximum tolerated dose of docetaxel when administered every three weeks is about 60 mg/m². In other embodiments, the cumulative maximum tolerated dose of docetaxel when administered every week is about 120 mg/m² or more, or about 40 mg/m²×3 or more.

In certain embodiments, a contemplated suspension is administered weekly for three weeks, followed by a week of no treatment. For example, provided herein is a method of treating a solid tumor cancer in a patient need thereof, comprising sequentially administering to the patient a docetaxel nanoparticle suspension having between about 35 mg/m² and about 45 mg/m² of docetaxel (e.g. 40 mg/m²) for a period of time, wherein the sequential administration is followed by a rest period of time, wherein the docetaxel nanoparticle suspension comprises:

a plurality of therapeutic nanoparticles comprising: docetaxel; poly(lactic) acid-poly(ethylene)glycol copolymer comprising poly(lactic acid) having a number average molecular weight of about 16 kDa and poly(ethylene)glycol having a number average molecular weight of about 5 kDa; a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and a surfactant; and an aqueous suspending medium. For example, the sequentially administrating may be repeated at least once. In some embodiments, the docetaxel nanoparticle suspension may be administered weekly for three weeks (e.g., sequentially administering a docetaxel nanoparticle suspension having about 40 mg/m² of docetaxel weekly for three weeks), followed by a seven day rest period of time.

Provided herein is a regimen for treating solid tumor cancers in a human patient, said regimen comprising delivering to the patient a therapeutic nanoparticle suspension in a monthly cycle of treatment, said monthly cycle comprising intravenously administering a first dosage of the therapeutic nanoparticle suspension comprising about 35 mg/m² and about 45 mg/m² docetaxel per week for at least one week in the cycle, followed by at least one week where no therapeutic nanoparticle suspension is administered, wherein the therapeutic nanoparticle suspension comprises: a plurality of therapeutic nanoparticles comprising: docetaxel; poly(lactic) acid-poly(ethylene)glycol copolymer comprising poly(lactic acid) having a number average molecular weight of about 16 kDa and poly(ethylene)glycol having a number average molecular weight of about 5 kDa; a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and a surfactant; and an aqueous suspending medium. For example, a monthly cycle comprises three weekly dosage administrations

In certain embodiments, the cancer treated by the disclosed methods and therapeutic nanoparticles is at least one of: breast, prostate, adenocarcinoma, non-small cell lung cancer, or ovarian cancer.

In certain embodiments, a contemplated suspension is administered once weekly at a dose of about 40 mg/m² of docetaxel.

In certain embodiments, a contemplated suspension is administered once weekly for three weeks and wherein the suspension is not administered during the fourth week.

In certain embodiments, a one month cycle of treatment of a contemplated suspension comprises once weekly treatment at 40 mg/m² for three weeks and one week with no treatment.

In certain embodiments, the average weekly dose of docetaxel is 30 mg/m².

In another aspect, a therapeutic nanoparticle is provided. The therapeutic nanoparticle comprises:

about 9 to 10 weight percent docetaxel;

about 80 to about 90 weight percent polylactic acid-polyethylene glycol block copolymer, wherein said poly(lactic) acid-poly(ethylene)glycol copolymer comprises poly(lactic acid) having a number average molecular weight of about 15 to 20 kDa and poly(ethylene)glycol having a number average molecular weight of about 4 to about 6 kDa; and

about 2 to about 3 weight percent of a targeting moiety represented by:

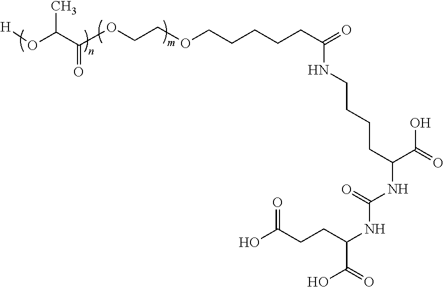

wherein n is about 200 to about 350 and m is about 110 to about 120. In certain embodiments, n is about 280 and m is about 115.

In certain embodiments, a contemplated therapeutic nanoparticle has a diameter of about 70 nm to about 130 nm. For example, in certain embodiments, a contemplated therapeutic nanoparticle has a diameter of about 100 nm.

In certain embodiments, a contemplated therapeutic nanoparticle further comprises about 5 to about 6 weight percent of a surfactant. For example, in certain embodiments, the surfactant is polysorbate 80.

In certain embodiments, a contemplated therapeutic nanoparticle has about 83 weight percent polylactic acid-polyethylene glycol block copolymer.

In certain embodiments, the poly(lactic) acid-poly(ethylene)glycol copolymer of a contemplated nanoparticle comprises poly(lactic acid) having a number average molecular weight of about 16 kDa and poly(ethylene)glycol having a number average molecular weight of about 5 kDa.

In still another aspect, a therapeutic nanoparticle suspension is provided. The therapeutic nanoparticle suspension comprises:

a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa with a pentylene end group, wherein the pentylene end group is conjugated through an amide linkage to the moiety S,S-2-{3-[1-carboxy-5-amino-pentyl]-ureido}-pentanedioic acid; and
a surfactant; and

an aqueous suspending medium. Such disclosed therapeutic nanoparticle suspensions may include concentrations of:

about 4.25 to about 5.75 mg/mL of the docetaxel;

about 46 mg/mL of the poly(lactic) acid-poly(ethylene)glycol copolymer;

about 1.2 mg/mL of the targeting polymer; and

about 3 mg/mL of the surfactant.

In certain embodiments, the surfactant in a contemplated suspension is polysorbate 80.

In certain embodiments, the aqueous suspending medium of a contemplated suspension comprises sucrose. For example, in certain embodiments, the aqueous suspending medium is about 32 weight percent sucrose and about 68 weight percent water.

In certain embodiments, a contemplated therapeutic nanoparticle suspension has a concentration of about 5 mg/mL of the docetaxel.

In certain embodiments, a contemplated therapeutic nanoparticle suspension has less than about 25 percent free docetaxel concentration.

In certain embodiments, the targeting polymer of a contemplated therapeutic nanoparticle is represented by:

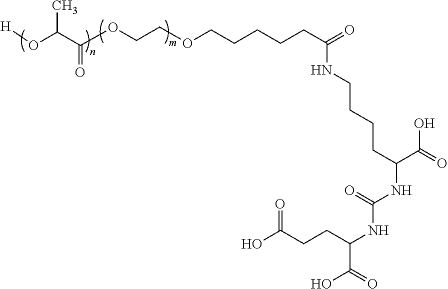

wherein n is about 280 and m is about 115.

### BRIEF DESCRIPTION OF THE DRAWINGS

FIGS. 1A-1C depict an exemplary synthetic scheme to a disclosed targeting polymer.

FIG. 2 is a flow chart for an emulsion process for forming disclosed nanoparticles.

FIGS. 3A and 3B show flow diagrams for a disclosed emulsion process. FIG. 3A shows particle formation and hardening (upstream processing), and FIG. 3B shows particle work up and purification (downstream processing).

FIG. 4 shows ¹H NMR spectra of disclosed nanoparticles having docetaxel (top spectrum) and polymer ligand and particles with no polymer ligand (bottom spectrum). (DMF internal standard concentration=300 μM for each sample.)

### DETAILED DESCRIPTION

The present disclosure generally relates to suspensions and compositions of polymeric nanoparticles that include docetaxel, as well as methods of treating various cancers, including refractory or drug resistant cancers in patients in need thereof using disclosed compositions.

Disclosed nanoparticles may include about 0.2 to about 35 weight percent, about 3 to about 40 weight percent, about 5 to about 12 weight percent, about 9 to about 11 weight percent, about 9 to about 10 weight percent, or about 9.5 weight percent of an active agent, such as antineoplastic agent, e.g. a taxane agent (for example docetaxel). For example, docetaxel anhydrous [(2R,3S)—N-carboxy-3-phenylisoserine, N-tert-butyl ester, 13-ester with 5-20-epoxy-1,2,4,7,10,13-hexahydroxytax-11-en-9-one 4-acetate 2-benzoate] may form part of a disclosed nanoparticle, and is a white to almost-white powder, practically insoluble in water, and has a specific optical rotation of −37.5° to −42.5° in methanol at a concentration of 10 mg/mL. The chemical formula of Docetaxel Anhydrous is C₄₃H₅₃NO₁₄. The molecular weight of Docetaxel Anhydrous is 807.9 g/mol. The active agent or drug may be a therapeutic agent such as an antineoplastic such as mTor inhibitors (e.g., sirolimus, temsirolimus, or everolimus), vinca alkaloids such as vincristine, a diterpene derivative or a taxane such as paclitaxel (or its derivatives such as DHA-paclitaxel or PG-paclitaxel).

Disclosed nanoparticles include PLA-PEG and a targeting polymer which comprises PLA-PEG conjugated to, i.e. covalently bound to a PMSA ligand such as disclosed herein, where the PLA-PEG may be bound via the PEG to the ligand through an alkylene (e.g., pentylene) linker. Poly-L-lactic acid, poly-D-lactic acid, poly-D,L-lactic acid, poly-L-lactide, poly-D-lactide, and poly-D,L-lactide, collectively referred to herein as “PLA.” In certain embodiments, the disclosed nanoparticles comprise about 10 to about 99 weight percent of biocompatible diblock poly(lactic) acid-poly(ethylene)glycol.

Particles disclosed herein include a polylactic acid-polyethylene glycol block copolymer (PLA-PEG) and a targeting polymer or moiety that includes a polylactic acid-polyethylene glycol block copolymer. It is contemplated that the PEG portion of either PLA-PEG portion may be terminated and/or include an end group, for example, when PEG is or is not conjugated to a ligand. For example, PEG may terminate in, or include, a hydroxyl, a methoxy or other alkoxyl group, a methyl or other alkyl group, an aryl group, (or an alkylene or phenylene group, e.g. a butylene, methylene, pentylene group that, when part of e.g. a targeting polymer, may be bound through an amide linkage to a PSMA targeting moiety.

Disclosed therapeutic nanoparticles may include a targeting moiety or targeting polymer. In certain embodiments, a low-molecular weight ligand such as a low-molecular weight PSMA ligand is conjugated to a PLA-PEG polymer, and the nanoparticle comprises a certain ratio of ligand-conjugated polymer (e.g., PLA-PEG-Ligand) to non-functionalized polymer (e.g. PLA-PEG). The ligand conjugated polymer may be a poly(lactic) acid-poly(ethylene)glycol polymer wherein the polylactic acid has a number average molecular weight of about 15 kDa to about 25 kDa (e.g., about 20 kDa), and the poly(ethylene)glycol has a number average molecular weight of about 5 kDa with a pentylene end group, where the pentylene end group is conjugated through an amide linkage to the moiety S,S-2-{3-[1-carboxy-5-amino-pentyl]-ureido}-pentanedioic acid.

Contemplated ligands conjugated to PLA-PEG to form e.g. a targeting polymer may include:

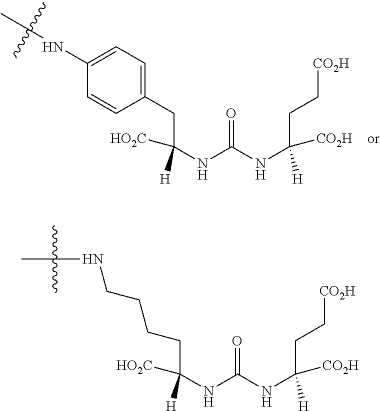 [SMILES: *Nc1ccc(C[C@H](NC(=O)N[C@@H](CCC(=O)O)C(=O)O)C(=O)O)cc1 ; *NCCCC[C@H](NC(=O)N[C@@H](CCC(=O)O)C(=O)O)C(=O)O]

For example disclosed nanoparticle may include a targeting moiety represented by:

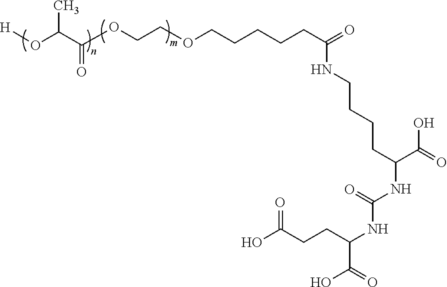

wherein n is about 200 to about 350 and m is about 105 to about 125, or n is about 250 to about 300 and m is about 110 to about 120, or n is about 280 and m is about 115. For example, provided herein is a therapeutic nanoparticle comprising:

about 8 to 11 weight percent, or about 9 to 10 weight percent, or about 9 to about 11 weight percent, e.g. about 9.5 weight percent docetaxel;

about 80 to about 90 weight percent polylactic acid-polyethylene glycol block copolymer, (or about 75 to about 90 weight percent polylactic acid-polyethylene glycol block copolymer, or about 80 to about 87, e.g. about 82, 83, 84, or 85 weight percent polylactic acid-polyethylene glycol block copolymer), wherein said poly(lactic) acid-poly(ethylene)glycol copolymer comprises poly(lactic acid) having a number average molecular weight of about 15 to 20 kDa (e.g. about 16 kDa) and poly(ethylene)glycol having a number average molecular weight of about 4 to about 6 kDa (e.g. about 5 kDa) and

a targeting moiety, for example about 1 to about 3 weight percent, or about 2 to about 3 weight percent of a targeting moiety, represented by:

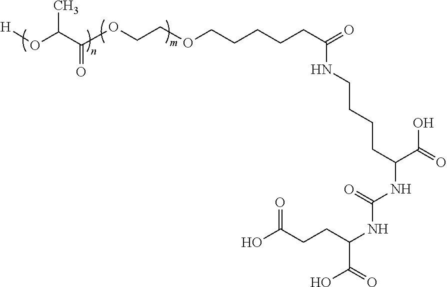

wherein n is about 200 to about 350 and m is about 110 to about 120, e.g., n is about 280 and m is about 115.

Contemplated therapeutic nanoparticles may have a diameter of about 70 nm to about 130 nm, about 80 nm to about 120 nm, e.g. a diameter of about 100 nm.

Disclosed nanoparticles may further comprise a surfactant or other excipient, e.g. may include about 5 to about 6 weight percent of a surfactant such a polysorbate 80.

In a specific embodiment, therapeutic nanoparticle is provided comprising:

about 9 to 10 weight percent docetaxel;

about 83 to about 84 weight percent polylactic acid-polyethylene glycol block copolymer, wherein said poly(lactic) acid-poly(ethylene)glycol copolymer comprises poly(lactic acid) having a number average molecular weight of about 16 kDa and poly(ethylene)glycol having a number average molecular weight of about 5 kDa;

about 5 to about 6 weight percent of a surfactant (e.g. polysorbate 80), and

about 2 to about 3 weight percent of a targeting moiety represented by:

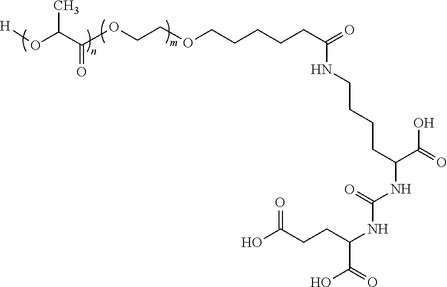

wherein n is about 280 and m is about 115.

Disclosed nanoparticles may be stable (e.g. retain substantially all active agent) for example in a solution that may contain a saccharide, for at least about 3 days, about 4 days or at least about 5 days at room temperature, or at 25° C.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

Nanoparticles may have controlled release properties, e.g., may be capable of delivering an amount of active agent to a patient, e.g., to specific site in a patient, over an extended period of time, e.g. over 1 day, 1 week, or more. In some embodiments, disclosed nanoparticles substantially immediately releases (e.g. over about 1 minute to about 30 minutes) less than about 2%, less than about 5%, or less than about 10% of an active agent (e.g. a taxane) agent, for example when places in a phosphate buffer solution at room temperature and/or at 37° C.

The pharmaceutical compositions of this invention can be administered to a patient by any means known in the art including oral and parenteral (e.g. intravenous) routes. The term “patient” or “subject” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles disclosed herein are administered to a subject in need thereof systemically, e.g., by IV infusion or injection.

In some embodiments, a therapeutic composition is provided that includes a plurality of disclosed nanoparticles in an aqueous composition. For example, such a composition may comprise disclosed nanoparticles in a medium that includes about 30 to about 40 weight percent disaccharide, e.g. sucrose, or for example about 32 weight percent sucrose and the balance water, e.g. about 68 weight percent water.

For example, provided herein is a therapeutic nanoparticle suspension comprising:

a plurality of therapeutic nanoparticles each substantially comprising:

docetaxel;

poly(lactic) acid-poly(ethylene)glycol block copolymer comprising poly(lactic acid) having a number average molecular weight of about 15 to 20 kDa, or about 16 kDa and poly(ethylene)glycol having a number average molecular weight of about 4-6 kDa, or about 5 kDa;

a targeting polymer comprising a poly(lactic) acid-poly(ethylene)glycol polymer with the poly(lactic) acid having a number average molecular weight of about 15 to about 25 kDa, or about 20 kDa, and poly(ethylene)glycol having a number average molecular weight of about 4 to 6 kDa, or about 5 kDa and having a pentylene end group, wherein the pentylene end group of the polyethylene glycol of the targeting polymer is conjugated through an amide linkage to the moiety S,S-2-{3-[1-carboxy-5-amino-pentyl]-ureido}-pentanedioic acid; and surfactant; and an aqueous suspending medium.

The targeting polymer may be, in certain embodiments, represented by:

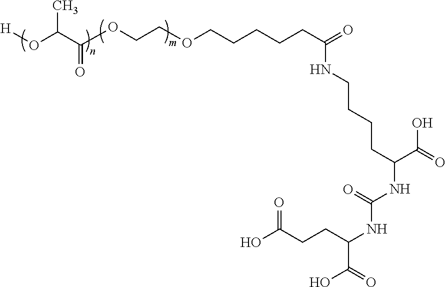

wherein n is about 280 and m is about 115.

Such disclosed therapeutic nanoparticle suspensions may include concentrations about 4.25 to about 5.75 mg/mL of the docetaxel; about 40-50 mg/mL, or about 45 to about 47 mg/mL, or about 46 mg/mL of the poly(lactic) acid-poly(ethylene)glycol block copolymer; about 1 to about 2 mg/mL, or about 1.1 to about 1.3 or about 1.2 mg/mL of the targeting polymer; and about 2-4 mg/mL or about 3 mg/mL of a surfactant (e.g. polysorbate 80).

In certain embodiments, the aqueous suspending medium comprises sucrose, e.g. about 30 to 35 weight percent or about 32 weight percent sucrose. In an embodiment, the aqueous suspending medium comprises about 68 weight percent water.

Disclosed therapeutic nanoparticle suspensions may have a concentration of about 4 mg/mL to about 6 mg/mL, e.g. about 5 mg/mL of the docetaxel. In certain embodiments, a contemplated therapeutic nanoparticle suspension may have less than about 20 percent, or less than about 25 percent free docetaxel concentration, e.g. docetaxel that is substantially unassociated with or unencapsulated by a nanoparticle of the suspension.

In some embodiments, targeted particles in accordance with the present invention may be used to treat, alleviate, ameliorate, relieve, delay onset of, inhibit progression of, reduce severity of, and/or reduce incidence of a cancer a patient or subject in need thereof. In some embodiments, inventive nanoparticles or compositions may be used to treat solid tumors, e.g., cancer and/or cancer cells. In certain embodiments, disclosed nanoparticles and compositions may be used to treat any cancer wherein PSMA is expressed on the surface of cancer cells or in the tumor neovasculature in a subject in need thereof, including the neovasculature of prostate or non-prostate solid tumors. Examples of the PSMA-related indication include, but are not limited to, prostate cancer, breast cancer, non-small cell lung cancer, colorectal carcinoma, and glioblastoma. The subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

The term “cancer” includes pre-malignant as well as malignant cancers. Cancers include, but are not limited to, prostate, gastric cancer, colorectal cancer, skin cancer, e.g., melanomas or basal cell carcinomas, lung cancer, breast cancer, cancers of the head and neck, bronchus cancer, pancreatic cancer, urinary bladder cancer, brain or central nervous system cancer, peripheral nervous system cancer, esophageal cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. “Cancer cells” can be in the form of a tumor, exist alone within a subject (e.g., leukemia cells), or be cell lines derived from a cancer.

Cancer can be associated with a variety of physical symptoms. Symptoms of cancer generally depend on the type and location of the tumor. For example, lung cancer can cause coughing, shortness of breath, and chest pain, while colon cancer often causes diarrhea, constipation, and blood in the stool. However, to give but a few examples, the following symptoms are often generally associated with many cancers: fever, chills, night sweats, cough, dyspnea, weight loss, loss of appetite, anorexia, nausea, vomiting, diarrhea, anemia, jaundice, hepatomegaly, hemoptysis, fatigue, malaise, cognitive dysfunction, depression, hormonal disturbances, neutropenia, pain, non-healing sores, enlarged lymph nodes, peripheral neuropathy, and sexual dysfunction.

In one aspect of the invention, a method for the treatment of cancer (e.g. prostate or breast cancer) is provided. In some embodiments, the treatment of cancer comprises administering a therapeutically effective amount of a disclosed particle or composition to a subject in need thereof, in such amounts and for such time as is necessary to achieve the desired result. In certain embodiments of the present invention a “therapeutically effective amount” of an inventive targeted particle is that amount effective for treating, alleviating, ameliorating, relieving, delaying onset of, inhibiting progression of, reducing severity of, and/or reducing incidence of one or more symptoms or features of cancer.

In certain embodiments, cancers that can be treated, prevented or managed using the compounds and therapeutic methods provided herein include, but are not limited to: bladder cancer, brain cancer, breast cancer, cervical cancer, colon cancer (including colorectal cancer), esophageal cancer, head and neck cancer, leukemia, liver cancer, lung cancer (both small cell and non-small cell), lymphoma, melanoma, myeloma, neuroblastoma, ovarian cancer, pancreatic cancer, prostate cancer, renal cancer, sarcoma (including osteosarcoma), skin cancer (including squamous cell carcinoma), stomach cancer, testicular cancer, thyroid cancer, and uterine cancer. Contemplated methods include treating patients suffering from a cancer such as kidney, vulvar, lung (e.g., non-small cell lung cancer), hepatobiliary, pancreatic, appendicular, uterine, renal, adenocarcinoma, gastroesophageal, breast, urothelial, melanoma, and/or ampullary. The cancer can be relapsed or refractory or resistant to another treatment.

For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the cancer is resistant to more than one chemotherapeutic agent, e.g., the cancer is a multidrug resistant cancer. In one embodiment, the cancer is resistant to one or more of a platinum based agent, an alkylating agent, an anthracycline and a vinca alkaloid. In one embodiment, the cancer is resistant to one or more of a platinum based agent, an alkylating agent, a taxane and a vinca alkaloid.

In one embodiment, the composition is administered in combination with one or more additional anticancer agent, e.g., chemotherapeutic agent, e.g., a chemotherapeutic agent or combination of chemotherapeutic agents described herein, and radiation.

For example, provided herein is a method of treating cancer, or a refractory cancer in patient in need thereof, comprising intravenously administering to the patient an effective amount of a disclosed nanoparticle suspension. Exemplary cancers or refractory cancers include those above, and for example, breast, prostate, ovarian, and/or gastroesophageal. In some embodiments, a method of treating a refractory cancer (such as a refractory gastroesophageal or breast cancer is provided, wherein the patient, before administration of a disclosed nanoparticle suspension, has been previously treated with a first line regimen, and optionally a second line and/or a third line of treatment, with or without previous radiation treatment. For example, methods of treating various cancers are provided, where the patient has previously undergone radiation treatment, and/or a regimen of taxol (in solution form) and/or taxotere, and/or Adriamycin® and/or cyclophosphamide and/or carboplatin, and/or a second line treatment of e.g. 5-FU, leucovorin, oxaplatin, and/or GI152.

In some embodiments, contemplated methods include administering disclosed nanoparticles or suspension once every week, every two weeks, every three weeks or every four weeks, for example, every week. In certain embodiments, the suspension may be administered weekly for one, two or three weeks, followed by a week of no treatment or more of no treatment. A disclosed suspension, e.g. having a docetaxel amount about 5 mg/mL, may be administered in a dose of about 15 mg/m² to 50 mg/m² or more, or about 30 mg/m² to about 50 mg/m² or more of docetaxel.

In certain embodiments, the cumulative maximum tolerated dose of docetaxel when a disclosed suspension is administered is greater when administered weekly as compared to administering the same suspension every three weeks. For example, a cumulative maximum tolerated dose of docetaxel when a disclosed suspension is administered every three weeks may be about 60 mg/m², as compared to cumulative maximum tolerated dose of docetaxel when the same disclosed suspension is administered every week is about 120 mg/m² or more, or about 40 mg/m²×3 or more. In one embodiment, weekly dosing of the disclosed suspension results in a 50% increase in the average weekly exposure of docetaxel to a patient.

In some embodiments, a disclosed suspension is administered at escalating doses or the same dose of docetaxel, on e.g. a weekly basis. In some embodiments, the escalating doses comprise at least a first dose level and a second dose level. In some embodiments, the escalating doses comprise at least a first dose level, a second dose level, and a third dose level. In some embodiments, the doses further comprise a fourth dose level. In some embodiments, the doses comprise a first dose level, a second dose level, a third dose level, a fourth dose level and a fifth dose level. In some embodiments, six, seven, eight, nine and ten dose levels are contemplated.

In some embodiments, each dose level is no more than 67%, or no more than 50% of the immediately following dose level. In some embodiments, each dose level is no more than 33% of the immediately following dose level. In some embodiments, each dose level is no more than 20% of the immediately following dose level. In some embodiments, dose levels are separated by ½ log units. In some embodiments, dose levels are separated by 1 log unit. In other embodiments, the dose levels are equal.

In some embodiments, a first dose level (e.g., as measured by docetaxel present in a dose of a disclosed nanoparticle suspensions) administered to a patient is from about 1 mg/m² to about 40 mg/m² or about 3.5 mg/m² to about 40 mg/m² or about 10 mg/m² to about 30 mg/m². Such a first dose level may be for example, administered in a first week of a patient's dosing regimen. In some embodiments, a second dose level (e.g. administered in a second week of patient's dosing regimen) is from about 7 mg/m² to about 40 mg/m² or about 15 mg/m² to about 30 mg/m². In some embodiments, the third dose level is from about 15 mg/m² to about 40 mg/m² or about 15 mg/m² to about 45 mg/m². In other embodiments, each dose level is the same for each administration, e.g. about 15 mg/m² to about 45 mg/m², or about e.g. administered once weekly, for example, for three weeks.

In some embodiments the first, second, and third dose levels are administered to the subject in a 21 or 28 cycle, for example, each dose level is escalated, or remains constant, for the first three weeks with a one week no dose schedule. In some embodiments the first, second, or third dose levels are administered to the subject e.g. each week for about 1 to about 4, 5, or 6 weeks.

In some embodiments the first dose level is administered to the subject for 1 week, (e.g. once in week 1, for example on day 1), the second dose level is administered to the subject for 1 week (e.g. once in week 2, for example on day 8), and the third dose level is administered to the subject for 1 week (e.g. once in week 3 for example on day 15). In some embodiments, the first, second, and third dose level are about the same, e.g. about 15 mg/m² to about 45 mg/m², e.g about 40 mg/m². In an exemplary embodiment, no dose is administered in the 4^{th} week.

In some embodiments the first dose level is administered to the subject for 2 weeks, the second dose level is administered to the subject for 2 weeks, and the third dose level is administered to the subject for 2 weeks.

For example, provided herein is a method of treating a solid tumor cancer in a patient need thereof, comprising sequentially administering to the patient a docetaxel nanoparticle suspension, e.g. a disclosed nanoparticle suspension for example having between about 35 mg/m² and about 45 mg/m² of docetaxel, during period of time (e.g., administering one dose weekly, for e.g. one, two, three, four or more weeks), wherein the sequential administration is followed by a rest period of time (e.g. one week, two weeks, three weeks or more). Such sequentially administrating may be repeated at least once, twice, three, four or more times. For example, the docetaxel nanoparticle suspension may be administered weekly for three weeks, followed by a seven day rest period of time. Such a method may comprise sequentially administering a docetaxel nanoparticle suspension having about 40 mg/m² of docetaxel weekly for three weeks, followed by one week of a rest period with no administration of a disclosed composition.

Also provided herein is a regimen for treating solid tumor cancers in a human patient, said regimen comprising delivering to the patient a disclosed therapeutic nanoparticle suspension in a monthly cycle of treatment, said monthly cycle comprising intravenously administering a first dosage of the therapeutic nanoparticle suspension comprising, for example, about 35 mg/m² and about 45 mg/m² docetaxel per week for at least one week in the cycle, followed by at least one week where no therapeutic nanoparticle suspension is administered.

Also provided herein is a kit for the administration of a dosage regimen of a therapeutic nanoparticle suspension comprising:

a sufficient quantity of the therapeutic nanoparticle suspension to administer the therapeutic nanoparticle suspension according to the following dosage regimen: administering a dosage of the therapeutic nanoparticle suspension comprising about 30 mg/m² to about 40 mg/m², or 35 mg/m² to about 45 mg/m², or about 20 mg/m² to about 60 mg/m², or about 40 mg/m², docetaxel once a week for the first three weeks to a patient; not administering the therapeutic nanoparticle suspension to the patient in the fourth week; and optionally repeating the dosage regimen; and

optionally instructions to administer the therapeutic nanoparticle suspension according to the dosage regimen, wherein the therapeutic nanoparticle suspension comprises:

a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant; and

an aqueous suspending medium.

The invention now being generally described, it will be more readily understood by reference to the following examples which are included merely for purposes of illustration of certain aspects and embodiments of the present invention, and are not intended to limit the invention in any way.

### EXAMPLES

The invention now being generally described, it will be more readily understood by reference to the following examples which are included merely for purposes of illustration of certain aspects and embodiments, and are not intended to limit the invention in any way.

### Example 1

### Synthesis of a Low-Molecular Weight PSMA Ligand (GL2)

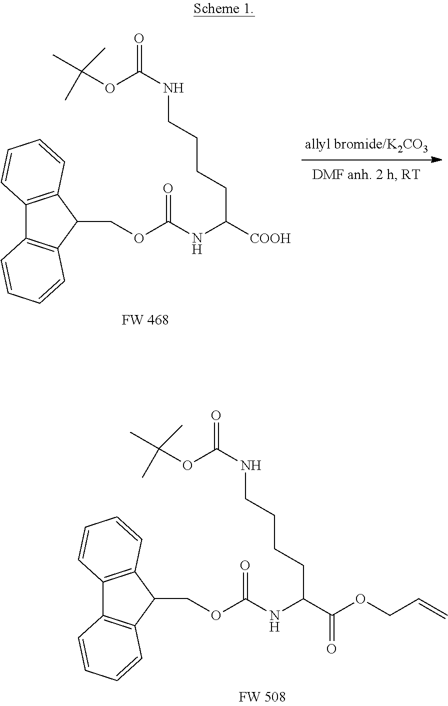 [SMILES: CC(C)(C)OC(=O)NCCCCC(NC(=O)OCC1c2ccccc2-c2ccccc21)C(=O)O ; C=CCOC(=O)C(CCCCNC(=O)OC(C)(C)C)NC(=O)OCC1c2ccccc2-c2ccccc21]

5 g (10.67 mmol) of the starting compound was dissolved in 150 mL of anhydrous DMF. To this solution was added allyl bromide (6.3 mL, 72 mmol) and K₂CO₃ (1.47 g, 10.67 mmol). The reaction was stirred for 2 h, the solvent was removed, the crude material was dissolved in AcOEt and washed with H₂O until pH neutral. The organic phase was dried with MgSO₄ (anhydrous) and evaporated to give 5.15 g (95%) of material. (TLC in CH₂Cl₂:MeOH 20:1 Rf=0.9, started compound Rf=0.1, revealed with ninhydrin and UV light).

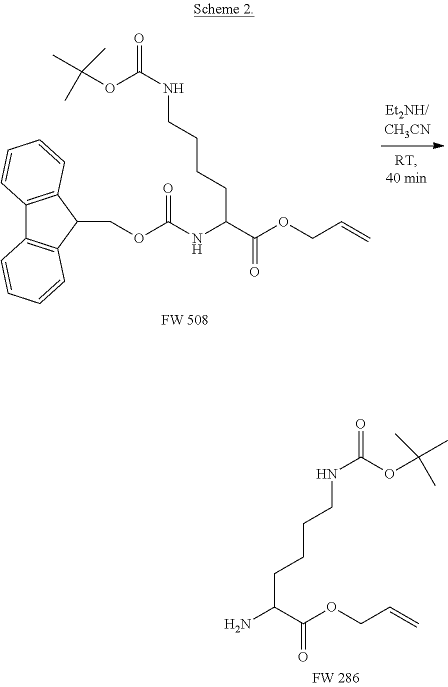 [SMILES: C=CCOC(=O)C(CCCCNC(=O)OC(C)(C)C)NC(=O)OCC1c2ccccc2-c2ccccc21 ; C=CCOC(=O)C(N)CCCCNC(=O)OC(C)(C)C]

To a solution of the compound (5.15 g, 10.13 mmol) in CH₃CN (50 mL) was added Et₂NH (20 mL, 0.19 mol). The reaction was stirred at room temperature for 40 min. The solvent was removed and the compound was purified by column chromatography (Hexane:AcOEt 3:2) to give 2.6 g (90%). (TLC in CH₂Cl₂:MeOH 10:1 Rf=0.4, revealed with ninhydrin (the compound has a violet color). ¹H-NMR (CDCl₃, 300 MHz) δ 5.95-5.85 (m, 1H, —CH₂CHCH₂), 5.36-5.24 (m, 2H, —CH₂CHCH₂), 4.62-4.60 (m, 3H, —CH₂CHCH₂, NHBoc), 3.46 (t, 1H, CH (Lys)), 3.11-3.07 (m, 2H, CH₂NHBoc), 1.79 (bs, 2H, NH₂), 1.79-1.43 (m, 6H, 3CH₂(Lys)), 1.43 (s, 9H, Boc).

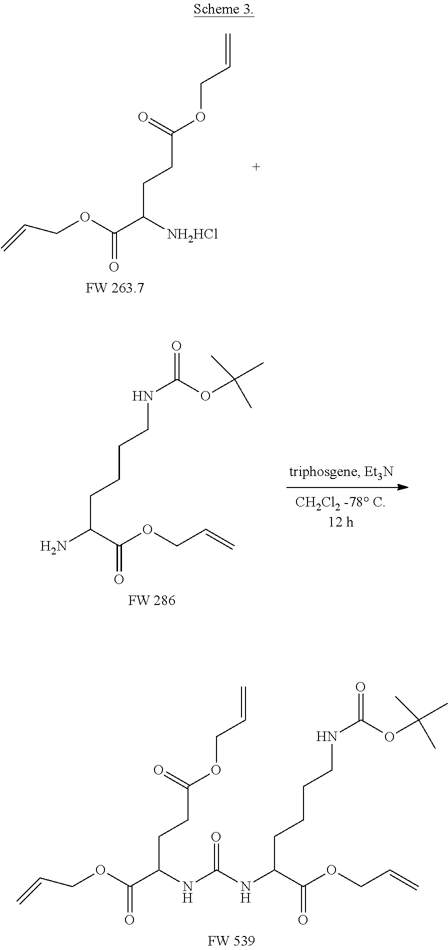

To a stirred solution of diallyl glutamate (3.96 g, 15 mmol) and triphosgene (1.47 g, 4.95 mmol) in CH₂Cl₂ (143 mL) at −78° C. was added Et₃N (6.4 mL, 46 mmol) in CH₂Cl₂ (28 mL). The reaction mixture was allowed to warm to room temperature and stirred for 1.5 h. The Lysine derivative (2.6 g, 9.09 mmol) in a solution of CH₂Cl₂ (36 mL) was then added at −78° C. and the reaction was stirred at room temperature for 12 h. The solution was diluted with CH₂Cl₂, washed twice with H₂O, dried over MgSO₄ (anh.) and purified by column chromatography (Hexane:AcOEt 3:1→2:1→AcOEt) to give 4 g (82%) (TLC in CH₂Cl₂:MeOH 20:1 Rf=0.3, revealed with ninhydrin). ¹H-NMR (CDCl₃, 300 MHz) δ 5.97-5.84 (m, 3H, 3-CH₂CHCH₂), 5.50 (bt, 2H, 2N Hurea), 5.36-5.20 (m, 6H, 3-CH₂CHCH₂), 4.81 (bs, 1H, NHBoc), 4.68-4.40 (m, 8H, 3-CH₂CHCH₂, CH (Lys), CH (glu)), 3.09-3.05 (m, 2H, CH₂NHBoc), 2.52-2.39 (m, 2H, CH₂ (glu.)), 2.25-2.14 and 2.02-1.92 (2m, 2H, CH₂ (glu.)), 1.87-1.64 (m, 4H, 2CH₂(Lys)), 1.51-1.35 (m, 2H, CH₂(Lys)), 1.44 (s, 9H, Boc).

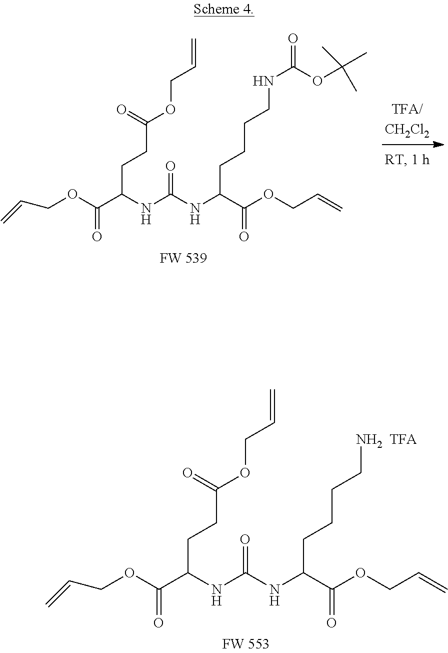

To a solution of the compound (4 g, 7.42 mmol) in dry CH₂Cl₂ (40 mL) was added at 0° C. TFA (9 mL). The reaction was stirred at room temperature for 1 h. The solvent was removed under vacuum until complete dryness, to give 4.1 g (quantitative). (TLC in CH₂Cl₂:MeOH 20:1 Rf=0.1, revealed with ninhydrin). ¹H-NMR (CDCl₃, 300 MHz) δ 6.27-6.16 (2d, 2H, 2N Hurea), 5.96-5.82 (m, 3H, 3-CH₂CHCH₂), 5.35-5.20 (m, 6H, 3-CH₂CHCH₂), 4.61-4.55 (m, 6H, 3-CH₂CHCH₂), 4.46-4.41 (m, 2H, CH (Lys), CH (glu)), 2.99 (m, 2H, CH₂NHBoc), 2.46 (m, 2H, CH₂ (glu.)), 2.23-2.11 and 2.01-1.88 (2m, 2H, CH₂ (glu.)), 1.88-1.67 (m, 4H, 2CH₂(Lys)), 1.45 (m, 2H, CH₂(Lys)).

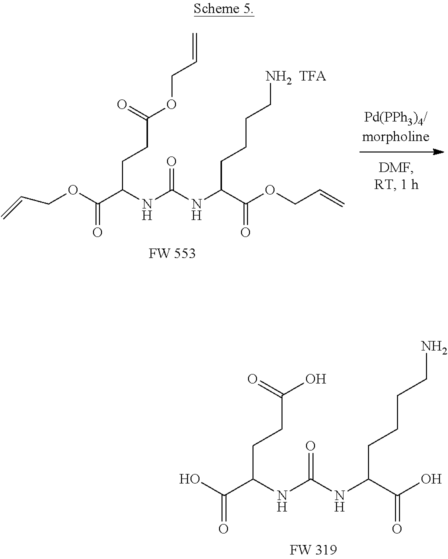

To a solution of the compound (2 g, 3.6 mmol) in DMF (anh.) (62 mL) under argon was added Pd(PPh₃)₄ (0.7 g, 0.6 mmol) and morpholine (5.4 mL, 60.7 mmol) at 0° C. The reaction was stirred at room temperature for 1 h. The solvent was removed. The crude product was washed twice with CH₂Cl₂, and then solved in H₂O. To this solution was added a diluted solution of NaOH (0.01 N) until the pH was very basic. The solvent was removed under reduced pressure. The solid was washed again with CH₂Cl₂, AcOEt, and a mixture of MeOH—CH₂Cl₂ (1:1), solved in H₂O and neutralized with Amberlite IR-120H⁺ resin. The solvent was evaporated, and the compound was precipitated with MeOH, to give 1 g (87%) of GL2. ¹H-NMR (D₂O, 300 MHz) δ 4.07 (m, 2H, CH (Lys), CH (glu)), 2.98 (m, 2H, CH₂NH₂), 2.36 (m, 2H, CH₂ (glu.)), 2.08-2.00 (m, 1H, CH₂ (glu)), 1.93-1.60 (m, 5H, CH₂ (glu.), 2CH₂(Lys)), 1.41 (m, 2H, CH₂(Lys)). Mass ESI: 320.47 [M+H⁺], 342.42 [M+Na⁺].

### Example 2

### Preparation of PLA-PEG

The synthesis is accomplished by ring opening polymerization of d,l-lactide with α-hydroxy-ω-methoxypoly(ethylene glycol) as the macro-initiator, and performed at an elevated temperature using Tin (II) 2-Ethyl hexanoate as a catalyst, as shown below (PEG Mn≈5,000 Da; PLA Mn≈16,000 Da; PEG-PLA Mₙ≈21,000 Da).

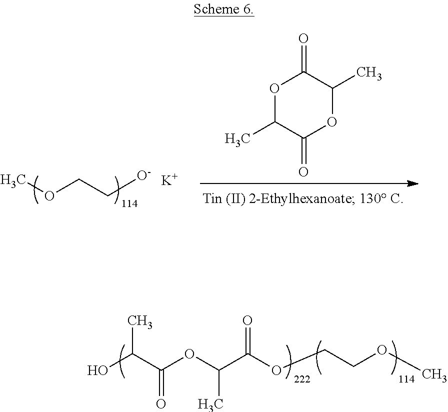

The polymer is purified by dissolving the polymer in dichloromethane, and precipitating it in a mixture of hexane and diethyl ether. The polymer recovered from this step shall be dried in an oven.

### Example 3

### PLA-PEG-Ligand Preparation

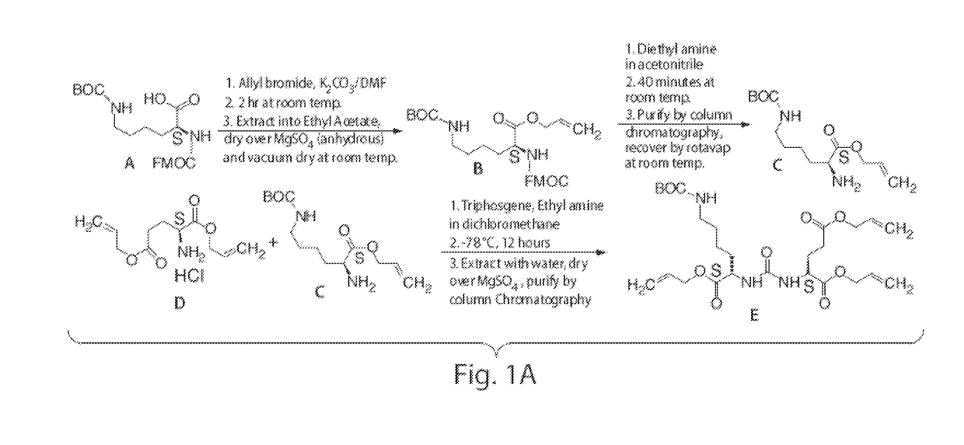

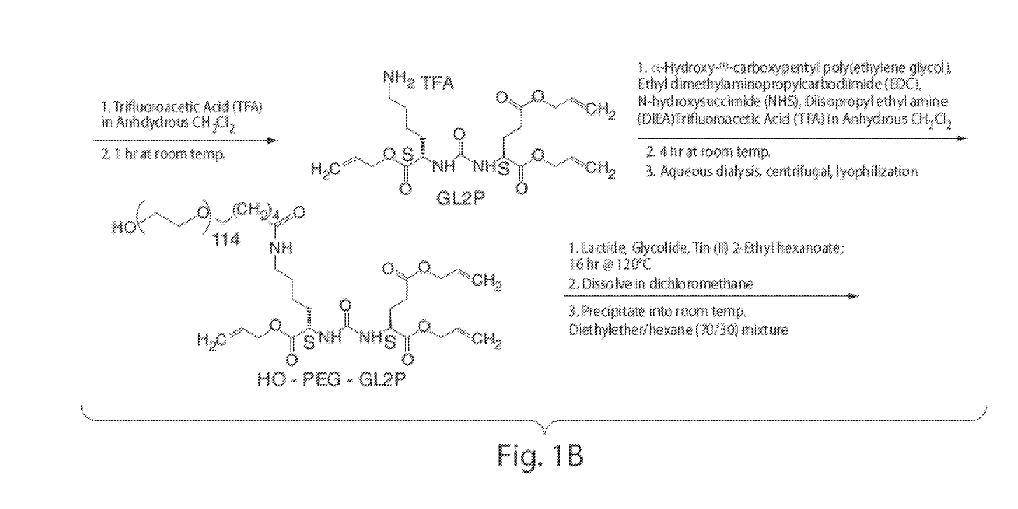

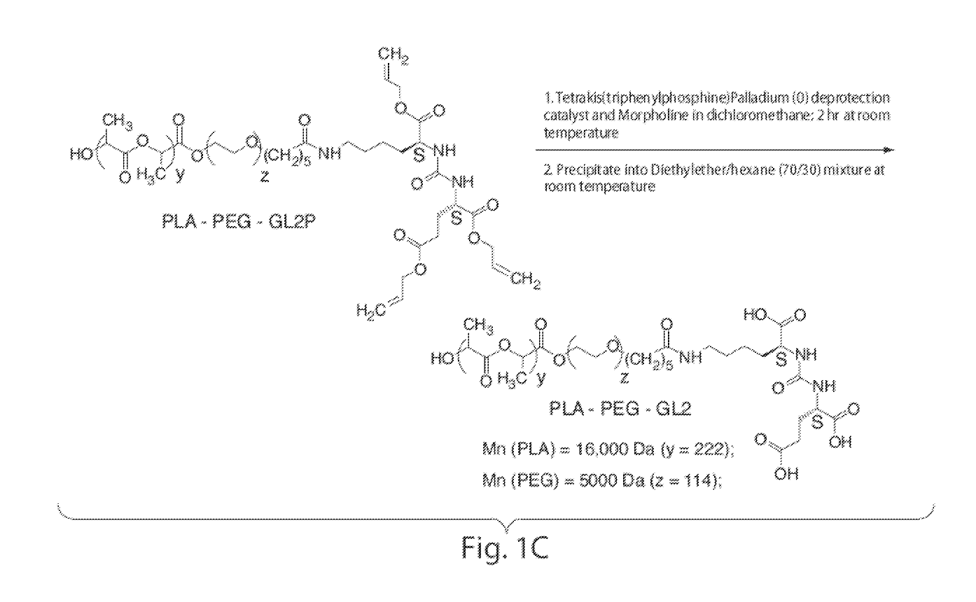

The synthesis, shown in FIGS. 1A-1C, starts with the conversion of FMOC, BOC lysine to FMOC, BOC, Allyl lysine by reacting the FMOC, BOC lysine with allyl bromide and potassium carbonate in dimethyl formamide, followed by treatment with diethyl amine in acetonitrile. The BOC, Allyl lysine is then reacted with triphosgene and diallyl glutamate, followed by treatment with trifluoroacetic acid in methylene chloride to form the compound “GL2P”.

The side chain amine of lysine in the GL2P is then PEGylated by the addition of Hydroxyl-PEG-Carboxylic acid with EDC and NHS. The conjugation of GL2P to PEG is via an amide linkage. The structure of this resulting compound is labeled “HO-PEG-GL2P”. Following the PEGylation, ring opening polymerization (ROP) of d,l-lactide with the hydroxyl group in the HO-PEG-GL2P as initiator is used to attach a polylactide block polymer to HO-PEG-GL2P via an ester bond yielding “PLA-PEG-GL2P”. Tin (II) 2-Ethyl hexanoate is used as a catalyst for the ring opening polymerization.

Lastly, the allyl groups on the PLA-PEG-GL2P are removed using morpholine and tetrakis(triphenylphosphine) palladium (as catalyst) in dichloromethane, to yield the final product PLA-PEG-Ligand. The final compound is purified by precipitation in 30/70% (v/v) diethyl ether/hexane.

### Example 4

### Nanoparticle Preparation—Emulsion Process

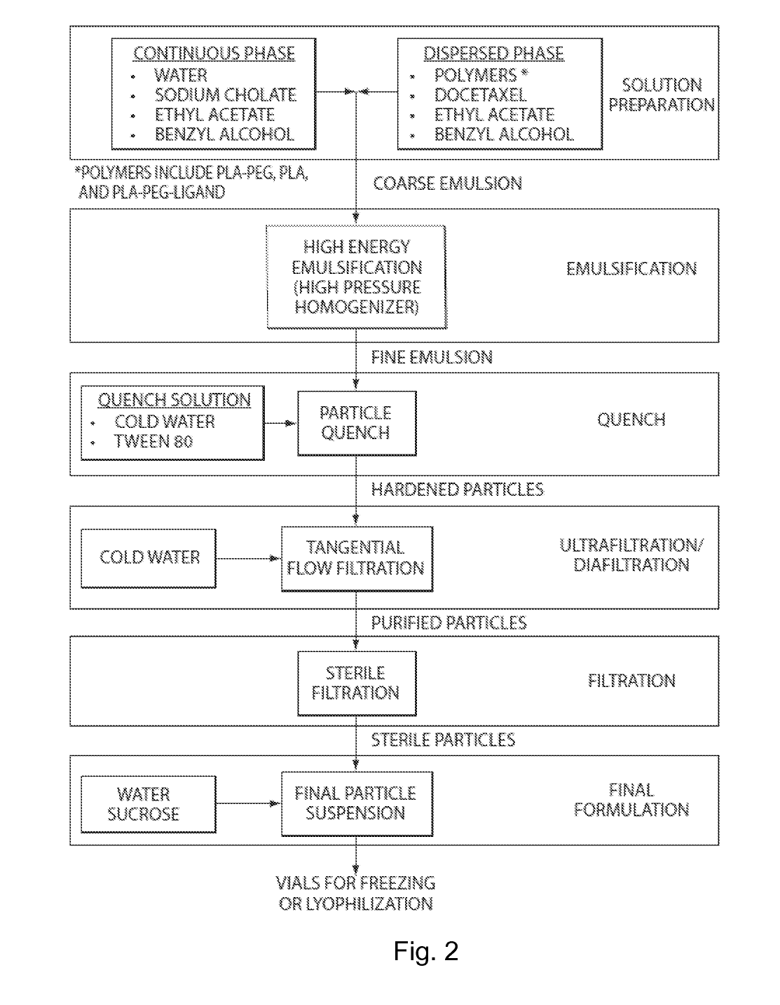

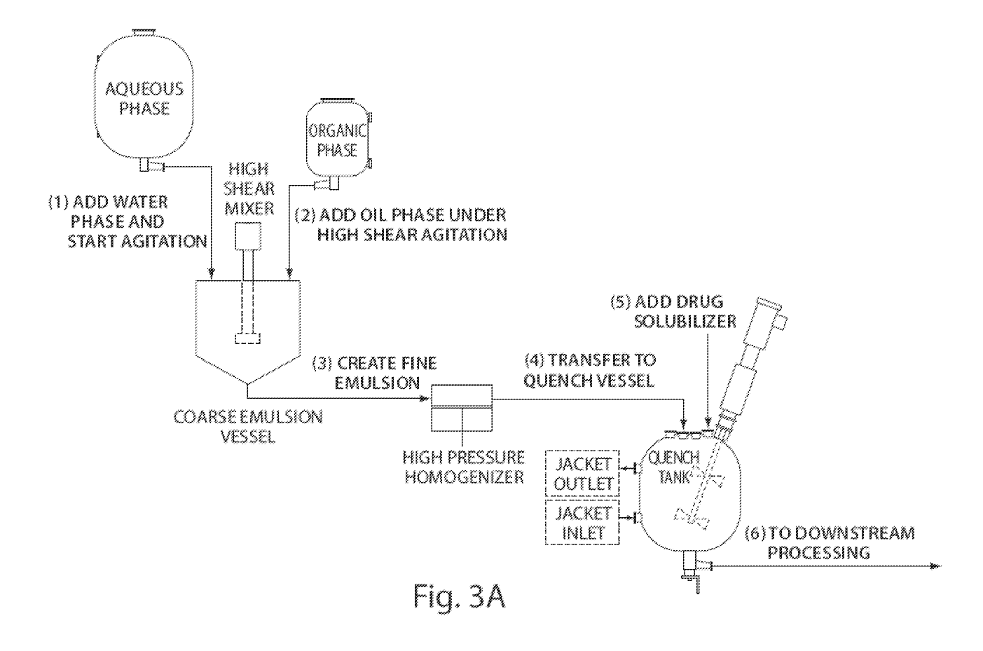

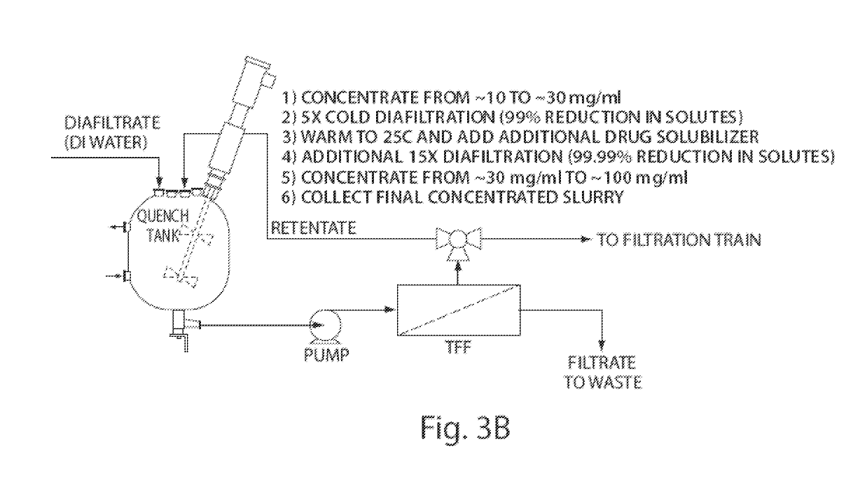

An organic phase is formed composed of a mixture of docetaxel (DTXL) and polymer (homopolymer, co-polymer, and co-polymer with ligand). The organic phase is mixed with an aqueous phase at approximately a 1:5 ratio (oil phase:aqueous phase) where the aqueous phase is composed of a surfactant and some dissolved solvent. In order to achieve high drug loading, about 30% solids in the organic phase is used. FIGS. 2, 3A, and 3B pictorially indicate the process below.

The primary, coarse emulsion is formed by the combination of the two phases under simple mixing or through the use of a rotor stator homogenizer. The rotor/stator yielded a homogeneous milky solution, while the stir bar produced a visibly larger coarse emulsion. It was observed that the stir bar method resulted in significant oil phase droplets adhering to the side of the feed vessel, suggesting that while the coarse emulsion size is not a process parameter critical to quality, it should be made suitably fine in order to prevent yield loss or phase separation. Therefore the rotor stator is used as the standard method of coarse emulsion formation, although a high speed mixer may be suitable at a larger scale.

The primary emulsion is then formed into a fine emulsion through the use of a high pressure homogenizer. The size of the coarse emulsion does not significantly affect the particle size after successive passes (103) through the homogenizer. M-110-EH.

Homogenizer feed pressure was found to have a significant impact on resultant particle size. On both the pneumatic and electric M-110EH homogenizers, it was found that reducing the feed pressure also reduced the particle size. Therefore the standard operating pressure used for the M-110EH is 4000-5000 psi per interaction chamber, which is the minimum processing pressure on the unit. The M-110EH also has the option of one or two interaction chambers. It comes standard with a restrictive Y-chamber, in series with a less restrictive 200 μm Z-chamber. It was found that the particle size was actually reduced when the Y-chamber was removed and replaced with a blank chamber. Furthermore, removing the Y-chamber significantly increases the flow rate of emulsion during processing.

After 2-3 passes the particle size was not significantly reduced, and successive passes can even cause a particle size increase. Placebo organic phase consisted of 25.5% polymer stock of 50:50 16.5/5 PLA/PEG:8.2 PLA. Organic phase was emulsified 5:1 O:W with standard aqueous phase, and multiple discreet passes were performed, quenching a small portion of emulsion after each pass. The indicated scale represents the total solids of the formulation.

The effect of scale on particle size showed surprising scale dependence. The trend shows that in the 2-10 g batch size range, larger batches produce smaller particles. It has been demonstrated that this scale dependence is eliminated when considering greater than 10 g scale batches. The amount of solids used in the oil phase was about 30%. For placebo batches the value for % solids represents the % solids were drug present at the standard 20% w/w. Table A summarizes the emulsification process parameters.

TABLE A

| Parameter | Value | Observation |
|---|---|---|
| Coarse | Rotor stator | Coarse emulsion size does not affect final |
| emulsion | homogenizer | particle size, but large coarse emulsion |
| formation | | can cause increased oil phase retention |
| | | in feed vessel |
| Homogenizer | 4000-5000 | Lower pressure reduces particle size |
| feed | psi per | |
| pressure | chamber | |
| Interaction | 2 × 200 μm | 200 μm Z-chamber yields the smallest |
| chamber(s) | Z-chamber | particle size, and allows for highest |
| | | homogenizer throughput |
| Number of | 2-3 passes | Studies have shown that the particle size |
| homogenizer | | is not significantly reduced after 2 discreet |
| passes | | passes, and size can even increase with |
| | | successive passes |
| Water phase | 0.1% | [Sodium cholate] can effectively alter |
| [sodium | | particle size; value is optimized for given |
| cholate] | | process and formulation |
| W:O ratio | 5:1 | Lowest ratio without significant particle |
| | | size increase is ~5:1 |
| [Solids] | 30% | Increased process efficiency, increased |
| in oil phase | | drug encapsulation, workable viscosity |

The fine emulsion is then quenched by addition to deionized water at a given temperature under mixing. In the quench unit operation, the emulsion is added to a cold aqueous quench under agitation. This serves to extract a significant portion of the oil phase solvents, effectively hardening the nanoparticles for downstream filtration. Chilling the quench significantly improved drug encapsulation. The quench:emulsion ratio is approximately 5:1.

A solution of 35% (wt %) of Tween 80 is added to the quench to achieve approximately 2% Tween 80 overall. After the emulsion is quenched a solution of Tween-80 is added which acts as a drug solubilizer, allowing for effective removal of unencapsulated drug during filtration. Table B indicates each of the quench process parameters.

TABLE B
Summary quench process parameters.

| Parameter | Value | Observation |
|---|---|---|
| Initial | <5° C. | Low temperature yields higher drug |
| quench | | encapsulation |
| temperature | | |
| [Tween-80] | 35% | Highest concentration that can be prepared |
| solution | | and readily disperses in quench |
| Tween-80: | 25:1 | Minimum amount of Tween-80 required to |
| drug ratio | | effectively remove unencapsulated drug |
| Q:E ratio | 5:1 | Minimum Q:E ratio while retaining high |
| | | drug encapsulation |
| Quench | ≦5° C. (with | Temperature which prevents significant |
| hold/ | current 5:1 | drug leaching during quench hold time |
| processing | Q:E ratio, 25:1 | and initial concentration step |
| temp | Tween-80: | |
| | drug ratio) | |

The temperature must remain cold enough with a dilute enough suspension (low enough concentration of solvents) to remain below the T_{g} of the particles. If the Q:E ratio is not high enough, then the higher concentration of solvent plasticizes the particles and allows for drug leakage. Conversely, colder temperatures allow for high drug encapsulation at low Q:E ratios (to ˜3:1), making it possible to run the process more efficiently.

The nanoparticles are then isolated through a tangential flow filtration process to concentrate the nanoparticle suspension and buffer exchange the solvents, free drug, and drug solubilizer from the quench solution into water. A regenerated cellulose membrane is used with a molecular weight cutoff (MWCO) of 300.

A constant volume diafiltration (DF) is performed to remove the quench solvents, free drug and Tween-80. To perform a constant-volume DF, buffer is added to the retentate vessel at the same rate the filtrate is removed. The process parameters for the TFF operations are summarized in Table C. Crossflow rate refers to the rate of the solution flow through the feed channels and across the membrane. This flow provides the force to sweep away molecules that can foul the membrane and restrict filtrate flow. The transmembrane pressure is the force that drives the permeable molecules through the membrane.

TABLE C
TFF Parameters

| | Optimized | | |
|---|---|---|---|
| Parameter | Value | | Effect |
| Membrane | Regenerated | | No difference in performance between RC and PES, but |
| Material | cellulose - | | solvent compatibility is superior for RC. |
| | Coarse Screen | | |
| | Membrane | | |
| Molecular | 300 | kDa | No difference in NP characteristics (i.e. residual |
| Weight | | | tween)Increase in flux rates is seen with 500 kDa |
| Cut off | | | membrane but 500 kDa is not available in RC |
| Crossflow | 11 | L/min/m² | Higher crossflow rate led to higher flux |
| Rate | | | |
| Transmembrane | 20 | psid | Open channel membranes have maximum flux rates |
| Pressure | | | between 10 and 30 psid. Coarse channel membranes have |
| | | | maximum flux rates with min TMP (~20 psid). |
| Concentration of | 30 | mg/ml | Diafiltration is most efficient at [NP] ~50 mg/ml with |
| Nanoparticle | | | open channel TFF membranes based on flux rates and |
| Suspension for | | | throughput. With coarse channel membranes the flux rate |
| Diafiltration | | | is optimized at ~30 mg/ml in the starting buffer. |
| Number of | ≧15 (based on | | About 15 diavolumes are needed to effectively remove |
| Diavolumes | flux increase) | | tween-80. End point of diafiltration is determined by in- |
| | | | process control (flux increase plateau). |
| Membrane | ~1 | m²/kg | Membranes sized based on anticipated flux rates and |
| Area | | | volumes required. |

The filtered nanoparticle slurry is then thermal cycled to an elevated temperature during workup. A small portion (typically 5-10%) of the encapsulated drug is released from the nanoparticles very quickly after its first exposure to 25° C. Because of this phenomenon, batches that are held cold during the entire workup are susceptible to free drug or drug crystals forming during delivery or any portion of unfrozen storage. By exposing the nanoparticle slurry to elevated temperature during workup, this ‘loosely encapsulated’ drug can be removed and improve the product stability at the expense of a small drop in drug loading. Table D summarizes two examples of 25° C. processing. Other experiments have shown that the product is stable enough after ˜2-4 diavolumes to expose it to 25° C. without losing the majority of the encapsulated drug. 5 diavolumes is used as the amount for cold processing prior to the 25° C. treatment.

TABLE D

| | | Lots A | Lots B |
|---|---|---|---|
| Drug load | Cold workup | 11.3% | 9.7% |
| | 25° C. workup¹ | 8.7-9.1% | 9.0-9.9% |
| Stability² | Cold workup | <1 day | <1 day |
| | 25° C. workup¹ | 5-7 days | 2-7 days |
| In vitro burst³ | Cold workup | ~10% | Not |
| | 25° C. workup¹ | ~2% | performed |

¹25° C. workup sublots were exposed to 25° C. after at least 5 diavolumes for various periods of time. Ranges are reported because there were multiple sublots with 25° C. exposure.
²Stability data represents the time that final product could be held at 25° C. at 10-50 mg/ml nanoparticle concentrations prior to crystals forming in the slurry (visible by microscopy)
³In vitro burst represents the drug released at the first time point (essentially immediately)

After the filtration process the nanoparticle suspension is passed through a sterilizing grade filter (0.2 μm absolute). Pre-filters are used to protect the sterilizing grade filter in order to use a reasonable filtration area/time for the process. Values are as summarized in Table E.

TABLE E

| Parameter | O Value | | Effect |
|---|---|---|---|
| Nanoparticle | 50 | mg/ml | Yield losses are higher at higher |
| Suspension | | | [NP], but the ability to filter |
| Concentration | | | at 50 mg/ml obviates the need to |
| | | | aseptically concentrate after filtration |
| Filtration | ~1.3 | L/min/m² | Filterability decreases as flow rate |
| flow rate | | | increases |

The filtration train is Ertel Alsop Micromedia XL depth filter M953P membrane (0.2 μm Nominal); Pall SUPRAcap with Seitz EKSP depth filter media (0.1-0.3 μm Nominal); Pall Life Sciences Supor EKV 0.65/0.2 micron sterilizing grade PES filter. 0.2 m² of filtration surface area per kg of nanoparticles for depth filters and 1.3 m2 of filtration surface area per kg of nanoparticles for the sterilizing grade filters can be used.

### Example 5

### Nanoparticle Suspension

A nanoparticle suspension (“Composition A”) is prepared that is a sterile, aqueous, particle suspension for IV administration containing docetaxel physically encapsulated in a polymer matrix composed of the biodegradable and biocompatible polymers PLA-PEG and PLA-PEG-GL. The particles are suspended in an aqueous sucrose solution.

The PLA-PEG-GL polymer is the PSMA targeting component of Composition A. The polymer is PLA-PEG that is end-functionalized with S,S-2-{3-[1-carboxy-5-amino-pentyl]-ureido}-pentanedioic acid (GL), a heterodimer comprising L-glutamic acid and L-lysine coupled by a urea linkage. The GL moiety is attached to the PEG terminus through an amide linkage to the lysine side chain amine. The PEG segment (number average molecular weight, 5,000 Da) is linked to PLA (20,000 Da) through an ester bond. The molecular weight of GL is 319 Da.

Poly(D,L-lactide-b-ethylene glycol) (PLA-PEG) is a biocompatible diblock copolymer, the constituents of which are approved for human use in both drug products and medical devices. The formula of PLA-PEG is HO(C₃H₄O₂)_{y}—(C₂H₄O)_{z}CH₃. The number average molecular weight of PLA is 16,000 Da, the number average molecular weight of PEG is 5,000 Da, and the number average molecular weight of PLA-PEG is 21,000 Da.

The components used in the manufacture of Composition A are presented in alphabetical order in Table F and manufacture was performed as described in Example 4.

TABLE F

| | | NOMINAL | | AMOUNT | |
|---|---|---|---|---|---|
| | | AMOUNT | | USED FOR | |
| COMPONENT | ROLE | PER VIAL | | 3-KG LOT | |
| Benzyl alcohol | Processing aid | None | | 2.07 | kg |
| Docetaxel | Active | 50 | mg | 600 | g |
| Anhydrous | pharmaceutical | | | | |
| | ingredient | | | | |
| Ethyl acetate | Processing aid | None | | 6.73 | kg |
| PLA-PEG | Drug encapsulation | 460 | mg | 2.34 | kg |
| | Drug release | | | | |
| | Surface properties | | | | |
| PLA-PEG-GL ^{a} | PSMA targeting | 12 | mg | 60 | g |
| Polysorbate 80 | Processing aid | 30 | mg | 18.6 | kg |
| Sodium cholate | Processing aid | None | | 75 | g |
| Sucrose | Cryoprotectant | 3.42 | g | 12 | kg |
| Water for | Medium | 7.43 | g | 2,000 | kg |
| injection (WFI) | | | | | |

^{a} The amount of PLA-PEG-GL used to formulate the drug product is 2.5 mol % of PLA-PEG and PLA-PEG-GL.

The final composition (Composition A) having nanoparticles is presented in Table G. The nanoparticles are packaged in 30-mL clear glass vials containing 10 mL (11.4 g) of suspension at a docetaxel concentration of 5 mg/mL.

TABLE G
Composition A.

| | | |
|---|---|---|
| | | NOMINAL |
| COMPONENT | ROLE | CONCENTRATION |

PARTICLE COMPONENTS

| | | | |
|---|---|---|---|
| Docetaxel | Active pharmaceutical | 5 | mg/mL |
| | ingredient | | |
| PLA-PEG | Drug encapsulation | 46 | mg/mL |
| | Drug release | | |
| | Surface properties | | |
| PLA-PEG-GL | PSMA targeting | 1.2 | mg/mL |
| Polysorbate 80 | Processing aid | 3 | mg/mL |

SUSPENDING MEDIUM COMPONENTS

| | | |
|---|---|---|
| Sucrose | Cryoprotectant | 32 wt % of |
| | | suspending medium |
| Water for | Medium | 68 wt % of |
| Injection | | suspending medium |

### Example 6

### Surface Charge by Zeta Potential

Zeta potential was measured for the A nanoparticle suspensions of Example 5 using a dilute salt solution (1 mM KCl or NaCl) as the dispersing agent. The measurements were taken at 25° C. on a Brookhaven ZetaPALs instrument with a 35 mW solid state laser at 660 nm. The software (ZetaPALs version 2.5) used the Smoluchowski model to calculate the zeta potential (Hosokawa et al., 2007). The results showed that the surface charge of the disclosed nanoparticle particle was weakly negative, with a zeta potential of approximately −10 to −15 mV. Particles formulated with 0 to 10% PLA-PEG-GL all exhibited zeta potentials between −10 to −15 mV indicating that surface charge was not strongly influenced by the presence of the low levels of GL targeting ligand utilized in the nanoparticles of composition A.

### Example 7

### Nanoparticle Surface GL Analysis by ¹H NMR Spectroscopy

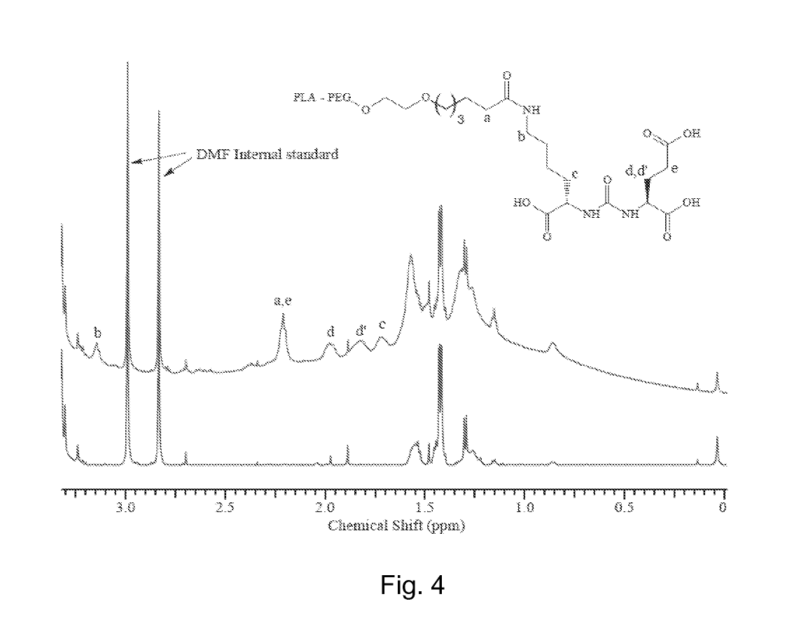

The presence of GL ligand at the particle surface of the particles of Example 5 was evaluated using ¹H NMR spectroscopy. The GL concentration is close to the detection limit of conventional NMR methods. To improve the sensitivity of the method for observing surface ligand, NMR spectra were acquired using a 600-MHz spectrometer. Samples were prepared using centrifugal filtration to exchange the particle storage solution (30% sucrose in water) with D₂O and to concentrate the suspension to a particle concentration of 100 mg/mL. Due to their significantly larger size relative to peaks associated with GL, signals from PEG and residual H₂O were suppressed using a pre-saturation technique. FIG. 4 shows spectra of composition A nanoparticles of example 6 and composition A-like nanoparticles composed of PLA-PEG and docetaxel but no PLA-PEG-GL. Well-resolved resonances assigned to GL ligand protons are indicated. The detection of ligand-associated resonances shows that the GL ligand is presented on the surface of the particle.

### Example 8

### Q3W Dosing

An open label, safety, pharmacokinetic and pharmacodynamic dose escalation study of using nanoparticle suspension of Example 5 was conducted. Nanoparticles were administered by intravenous (IV) infusion to patients with advanced or metastatic cancer. The study assessed the dose limiting toxicity (DLT) and maximum tolerated dose (MTD) of the composition when administered by IV once every 3 weeks on day 1 of a 21-day schedule (Q3W). The study also sought to characterize the pharmacokinetics of composition A following IV infusion along the Q3W schedule, to assess preliminary evidence of anti-tumor activity of Compound A using Response Evaluation Criteria in Solid Tumors (RECIST version 1.1) imaging evaluation, and to assess changes in serum tumor marker including: PSA, CA 125, CA 15-3 and CA 27.29, or CA 19-9.

Patients were enrolled into dose cohorts to receive IV doses of composition A (as in Example 5) on day 1 of a 21-day schedule (Q3W). Escalation to the next dose level was dependent on the incidence of DLTs observed within the first cycle administered to patients within each cohort. Doses were escalated until the MTD^{Q3W} were reached.

Each patient received one dose of composition A on Day 1 of Cycle 1. A cycle was defined as 21 days. Patients were treated with composition A on Day 1 of each additional cycle until they discontinued the study due to medical considerations or administrative considerations. Patients were pre-treated with corticosteroids and antihistamines.

Doses were escalated starting at 3.5 mg/m² (based on amount of docetaxel) until the MTD^{Q3W} was reached. The MTD was defined as the highest dose level that does not meet the definition of a DLT. The DLT dose level was defined as the lowest dose level at which a DLT was experienced in two or more patients out of a maximum of 6 patients in that dose group.

Accelerated escalation with 1 patient per dose level was continued until a patient had a grade ≧2 toxicity in his or her first cycle of treatment. Unless the grade ≧2 toxicity was clearly related to disease progression, the accelerated phase was terminated, and the non-accelerated phase began. A minimum of three evaluable patients were accrued at the dose that triggered the switch to the non-accelerated design and at each subsequent dose level. During the accelerated phase, no patient was enrolled at the next higher dose level until the patient at the current lower dose level was observed for at least 21 days (completed Cycle 1).

In the non-accelerated phase, if 1 of the 3 patients had a DLT, the cohort was expanded to a maximum of 6 patients. If only 1 of the 6 patients had a DLT, dose escalation continued. If two patients had a DLT, dose escalation stopped. The dose level at which 2 of 6 patients had a DLT was considered at least 1 dose level above the MTD. The next lower dose was then more fully evaluated by treating up to 6 patients. If 2 or more patients had DLTs at this lower dose level, de-escalation continued until a dose level was identified at which zero or only 1 of the initial 6 patients enrolled at that dose level had a DLT. This was identified as the MTD^{Q3W}. After the MTD was identified (the dose level at which zero or 1 of the initial 6 patients enrolled at that dose level had a DLT), a total of twelve patients (i.e. an additional six patients) were enrolled to further characterize adverse events and pharmacokinetics.

The following Table H illustrates the dose escalation scheme used in the Q3W trial.

TABLE H
Dose Escalation (Modified Fibonacci Design)

| Dose | % Increment from | |
|---|---|---|
| Level | Prior Dose Level | Composition A |
| 1 | — | 3.5 mg/m² |
| 2 | 100% | 7 mg/m² |
| 3 | 114% | 15 mg/m² |
| 4 | 100% | 30 mg/m² |
| 5 | 100% | 60 mg/m² |
| 6 | 25% | 75 mg/m² |
| 7 | 20% | 90 mg/m² |
| 8 | 22% | 110 mg/m² |

Dose groups received escalating doses of an IV infusion of the composition over 60 minutes (in either 0.9% sodium chloride solution or 5% dextrose solution) administered on Day 1 of a 21-day schedule (Q3W). Patients received standard premedication with corticosteroids and antihistamines. The MTD was determined to be 60 mg/m² as administered by IV once every 3 weeks on day 1 of a 21-day schedule (Q3W)

### Example 9

### Q1W Dosing

A open label, safety, pharmacokinetic and pharmacodynamic dose escalation study of the suspension of Example 5 was conducted. Nanoparticles in a composition A were administered by intravenous (IV) infusion to patients with advanced or metastatic cancer. The study was conducted to assess the dose limiting toxicity (DLT) and maximum tolerated dose (MTD) of Compound A when administered by IV once weekly on days 1, 8, and 15 of a 28-day schedule (Q1W). The study also sought to characterize the pharmacokinetics of the composition following IV infusion along the Q1W schedule, to assess preliminary evidence of anti-tumor activity of the composition using Response Evaluation Criteria in Solid Tumors (RECIST version 1.1) imaging evaluation, and to assess changes in serum tumor marker including: PSA, CA 125, CA 15-3 and CA 27.29, or CA 19-9.

Each patient received one dose of composition A on Days 1, 8, and 15 of Cycle 1. A cycle was defined as 28 days. Patients were treated with composition A on Days 1, 8, and 15 of each additional cycle until they discontinued the study. Patients were pre-treated with corticosteroids and antihistamines.

The starting dose used in the trial was 15 mg/m², which corresponds to a cumulative dose of 45 mg/m² within a 28-day period. Patients then were given subsequent incremental increases to 25, 30, 35, and 40-mg/m² dose levels.

A minimum of three patients were treated at each dose level. If 1 of the 3 patients had a DLT, the cohort was expanded to a maximum of 6 patients. If only 1 of the 6 patients had a DLT, dose escalation was continued. If 2 patients had a DLT, dose escalation was terminated. The dose level at which 2 of 6 patients had a DLT was considered at least 1 dose level above the MTD. The next lower dose was then more fully evaluated by treating up to 6 patients. If 2 or more patients had DLTs at this lower dose level, de-escalation continued until a dose level was identified at which zero or only 1 of the initial 6 patients enrolled at that dose level had a DLT. This was identified as the MTD^{Q1W}.

The following Table I illustrates the dose escalation scheme used in the Q1W trial.

TABLE I

| Dose | % Increment from | Composition A Dose (based |
|---|---|---|
| Level | Prior Dose Level | on docetaxel amount) |
| 1 | — | 15 mg/m² |
| 2 | 67% | 25 mg/m² |
| 3 | 20% | 30 mg/m² |
| 4 | 17% | 35 mg/m² |
| 5 | 14% | 40 mg/m² |
| 6 | 12.5% | 45 mg/m² |

Dose groups received escalating doses of an IV infusion of composition A over 60 minutes in 250 mL of either 0.9% sodium chloride solution or 5% dextrose solution, administered once weekly on Days 1, 8, and 15 of a 28-day schedule (Q1W). Patients received standard premedication with either oral administration of 8 mg dexamethasone, 1 hour before infusion, or IV administration of 8 mg dexamethasone before infusion. Table J provides information about the patient population, and Table K provides information on specific cancers of patients (PD: Progressive Disease; SD: Stable Disease).

TABLE J

| Characteristic | No. of Patients |
|---|---|
| Total Patients | 20 |
| Evaluable for Dose-Limiting Toxicities in Cycle 1 | 18 |
| Gender: male/female | 11/9 |
| Age, median (range), y | 61 (38-78) |

TABLE K

| Cancer | Dose Level | Cycles | |
|---|---|---|---|
| Diagnosis | (mg/m²) | Received | Response |
| Kidney | 15 | C2 | PD |
| Vulvar | 15 | C1 | PD |
| NSCLC | 15 | C2 | PD |
| Hepatobiliary | 25 | C2 | PD |
| Pancreas | 25 | C1 | PD |
| Appendicular | 25 | C1 | PD |
| Uterine | 30 | C1 | PD |
| Renal | 30 | C2 | PD |
| Adenocarcinoma | 30 | C2 | PD |
| (unknown) | | | |
| Gastroesophageal | 30 | C3+ | SD/uPR (↓33%) |
| Breast | 30 | C3+ | SD (↓8%) |
| NSCLC | 35 | C2 | PD |
| Mesothelioma | 35 | C2+ | |
| NSCLC | 35 | C1+ | |
| Pancreas | 35 | C1 | PD |
| Uterine | 35 | C1+ | |
| Urothelial | 40 | C1+ | |
| Melanoma | 40 | C1+ | |
| Hepatobiliary | 40 | C1+ | |
| Ampullary | 40 | C1+ | |

One patient enrolled in the trial suffering from gastroesophageal cancer responded to a 30 mg/m² dose of composition A. This patient had previously been treated with a first line regimen of taxol and carboplatin, a second line regimen of G1152, 5-FU, leucovorin, oxaliplatin, and ramucirumab, and a third line regimen of CPT111. The same patient had undergone radiation treatment targeted to the gastroesophageal junction. A second patient suffering from breast cancer also responded to a 30 mg/m² dose of composition A. This patient had previously been treated with a first line regimen of Taxotere®, Adriamycin®, and cyclophosphamide, and a second line regimen of tamoxifen. This patient had no prior exposure to radiation therapy.

### Example 10

An open label, safety, pharmacokinetic and pharmacodynamic dose escalation study of using nanoparticle suspension of Example 5 is designed. Nanoparticles are administered by intravenous (IV) infusion to patients with advanced or metastatic cancer. The study considers the composition when administered by IV once weekly on days 1, 8, and 15 of a 28-day schedule. The study also seeks to characterize the pharmacokinetics of composition A following IV infusion along the three week on/one week off schedule, to assess preliminary evidence of anti-tumor activity of Compound A using Response Evaluation Criteria in Solid Tumors (RECIST version 1.1) imaging evaluation, and to assess changes in serum tumor marker including: PSA, CA 125, CA 15-3 and CA 27.29, or CA 19-9. Patients are enrolled into dose cohorts to receive IV doses of composition A (as in Example 5) on day 1 of a 21-day schedule (Q3W).

Each patient received one dose (40 mg/m² of docetaxel via composition A on Day 1 of Cycle 1. A cycle is defined as 21 days (or 28 days with 7 days off). Patients can be pre-treated with corticosteroids and antihistamines.

Administration of composition A in patients may result in less neutropenia when administered the nanoparticle composition weekly for three weeks at/one week off at a dose of 40 mg/m² of docetaxel, e.g. as compared to administering 60 mg/m² of docetaxel once every three weeks. Weekly dosing may allow for greater drug exposure which potentially could have a positive effect on efficacy.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A method of treating a solid tumor cancer in patient in need thereof, comprising intravenously administering to the patient an effective amount of a therapeutic nanoparticle suspension comprising:
a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant; and
an aqueous suspending medium, wherein the suspension is administered to the patient once every week, every two weeks, every three weeks, or every four weeks.

2. A method of treating a solid tumor cancer in a patient need thereof, comprising sequentially administering to the patient a docetaxel nanoparticle suspension having between about 35 mg/m² and about 45 mg/m² of docetaxel for a period of time, wherein the sequential administration is followed by a rest period of time, wherein the docetaxel nanoparticle suspension comprises:
a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant; and
an aqueous suspending medium.

3. The method of claim 2, wherein the sequentially administrating is repeated at least once.

4. The method of claim 2 or 3, wherein the docetaxel nanoparticle suspension is administered weekly for three weeks, followed by a seven day rest period of time.

5. The method of any one of claims 2-4, comprising sequentially administering a docetaxel nanoparticle suspension having about 40 mg/m² of docetaxel weekly for three weeks.

6. A regimen for treating solid tumor cancers in a human patient, said regimen comprising delivering to the patient a therapeutic nanoparticle suspension in a monthly cycle of treatment, said monthly cycle comprising intravenously administering a first dosage of the therapeutic nanoparticle suspension comprising about 35 mg/m² and about 45 mg/m² docetaxel per week for at least one week in the cycle, followed by at least one week where no therapeutic nanoparticle suspension is administered, wherein the therapeutic nanoparticle suspension comprises:
a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant; and
an aqueous suspending medium.

7. The regimen according to claim 6, wherein the monthly cycle comprises three weekly dosage administrations.

8. The regimen according to claim 6, wherein the therapeutic nanoparticle suspension is delivered for two to twelve cycles.

9. The regimen according to claim 6, wherein the monthly cycles are continuous.

10. The method of any one of claims 1-9, wherein the solid tumor cancer is a refractory or relapsed cancer.

11. The method of any one of claims 1-10, wherein the solid tumor cancer is one or more of: breast, prostate, adenocarcinoma, non-small cell lung cancer, or ovarian cancer.

12. The method of claim 1, wherein the suspension is administered every week.

13. The method of claim 1, wherein the suspension is administered in a dose of about 15 mg/m² to 50 mg/m² or more, or about 30 mg/m² to about 50 mg/m² or more of docetaxel.

14. The method of claim 1, wherein the suspension is administered weekly for three weeks, followed by a week of no treatment.

15. The method of claim 13, wherein said suspension is administered once weekly at a dose of at least about 40 mg/m² of docetaxel.

16. The method claim 1, wherein administration comprises three step-wise increasing dose levels wherein each dose level is administered to the patient once a week for at least three weeks.

17. A kit for the administration of a dosage regimen of a therapeutic nanoparticle suspension comprising:
a sufficient quantity of the therapeutic nanoparticle suspension to administer the therapeutic nanoparticle suspension according to the following dosage regimen: administering a dosage of the therapeutic nanoparticle suspension comprising about 30 mg/m² to about 40 mg/m², or 35 mg/m² to about 45 mg/m², or about 40 mg/m², docetaxel once a week for the first three weeks to a patient; not administering the therapeutic nanoparticle suspension to the patient in the fourth week; and optionally repeating the dosage regimen; and
optionally instructions to administer the therapeutic nanoparticle suspension according to the dosage regimen, wherein the therapeutic nanoparticle suspension comprises:
a plurality of therapeutic nanoparticles comprising:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant; and
an aqueous suspending medium.

18. A therapeutic nanoparticle suspension comprising:
therapeutic docetaxel nanoparticles and an aqueous suspending medium, wherein the concentration of docetaxel in the suspension is about 4 mg/mL to about 6 mg/mL; and wherein the therapeutic docetaxel nanoparticles each comprise:
docetaxel;
poly(lactic) acid-poly(ethylene)glycol copolymer comprising poly(lactic acid) having a number average molecular weight of about 16 kDa and poly(ethylene)glycol having a number average molecular weight of about 5 kDa;
a targeting polymer comprising a poly(lactic) acid-poly(ethylene)glycol polymer with the poly(lactic) acid having a number average molecular weight of about 20 kDa and poly(ethylene)glycol having a number average molecular weight of about 5 kDa and having a pentylene end group, wherein the pentylene end group is conjugated through an amide linkage to the moiety S,S-2-{3-[1-carboxy-5-amino-pentyl]-ureido}-pentanedioic acid; and
a surfactant.

19. The therapeutic nanoparticle suspension of claim 18, wherein the suspension has a concentration of:
about 4.25 to about 5.75 mg/mL of the docetaxel;
about 46 mg/mL of the poly(lactic) acid-poly(ethylene)glycol copolymer;
about 1.2 mg/mL of the targeting polymer; and
about 3 mg/mL of the surfactant.

20. The therapeutic nanoparticle suspension of claim 18 or 19, wherein the surfactant is polysorbate 80.

21. The therapeutic nanoparticle suspension of any one of claims 18-20, wherein the aqueous suspending medium comprises sucrose.

22. The therapeutic nanoparticle suspension of any one of claims 18-21, wherein the aqueous suspending medium is about 32 weight percent sucrose and about 68 weight percent water.

23. The therapeutic nanoparticle suspension of any one of claims 18-22, wherein the suspension has a concentration of about 5 mg/mL of the docetaxel.

24. The therapeutic nanoparticle suspension of any one of claims 18-23, wherein the suspension has less than about 25 percent free docetaxel concentration.

25. The therapeutic nanoparticle suspension of any one of claims 18-24, wherein the targeting polymer is represented by: 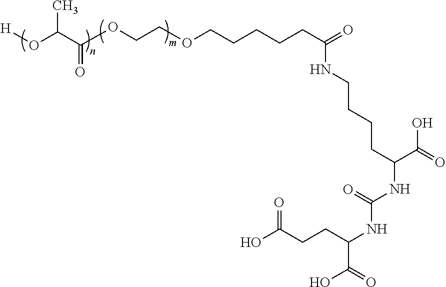
wherein n is about 280 and m is about 115.

26. A therapeutic nanoparticle comprising:
about 9 to 10 weight percent docetaxel;
about 80 to about 90 weight percent polylactic acid-polyethylene glycol block copolymer, wherein said poly(lactic) acid-poly(ethylene)glycol copolymer comprises poly(lactic acid) having a number average molecular weight of about 16 kDa and poly(ethylene)glycol having a number average molecular weight of about 5 kDa; and
about 2 to about 3 weight percent of a targeting moiety represented by: 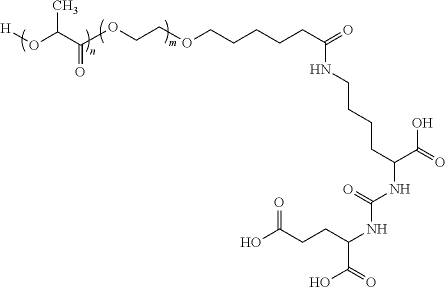
wherein n is about 200 to about 350 and m is about 110 to about 120.

27. The therapeutic nanoparticle of claim 26, wherein n is about 280 and m is about 115.

28. The therapeutic nanoparticle of claim 26 or 27, having a diameter of about 70 nm to about 130 nm.

29. The therapeutic nanoparticle of any one of claims 26-28, having a diameter of about 100 nm.

30. The therapeutic nanoparticle of any one of claims 26-29, wherein the nanoparticle further comprises about 5 to about 6 weight percent of polysorbate 80.

31. The therapeutic nanoparticle of any one of claims 26-30, having about 83 weight percent polylactic acid-polyethylene glycol block copolymer.
